(19) United States
(12) Patent Application Publication
(10) Pub. No.: US 2009/0155171 A1
(43) Pub. Date: Jun. 18, 2009

(54) VISUALIZING AGENT COMPRISING A JANUS GREEN B AND VISUALIZING METHOD BY USING THE SAME

(75) Inventors: Kwang-Sup Soh, Gyeonggi-do (KR); Byung-Cheon Lee, Gyeonggi-do (KR); Jung-Sun Yoo, Ulsan-city (KR); Ku-Youn Baik, Gyeonggi-do (KR); Sungil Cho, Seoul (KR)

Correspondence Address: LEXYOUME IP GROUP, LLC, 5180 PARKSTONE DRIVE, SUITE 175, CHANTILLY, VA, 20151, US

(73) Assignee: SEOUL NATIONAL UNIVERSITY INDUSTRY FOUNDATION, Seoul (KR)

(21) Appl. No.: 11/576,501

(22) PCT Filed: May 20, 2005
(86) PCT No.: PCT/KR2005/001484

§ 371 Date: Apr. 2, 2007

(51) Int. Cl. A61K 49/00; G01N 1/30

(52) U.S. Cl. 424/9.1; 435/40.5

(57) ABSTRACT

The present invention relates to a visualizing agent comprising a Janus Green B(JGB), and a visualizing method by using the same. More specifically, the method comprising A method of visualizing the intravascular threadlike structure (Bonghan duct) floating inside a lymphatic vessel comprising injecting JGB into the lymphatic tissue to be stained, and conducting microscopy. The present invention provides a major breakthrough in cellular anatomy and physiology. Further studies of its histological aspects and physiological functions suggest the possibility of critical new insights in both biology and medicine.

### BACKGROUND OF THE INVENTION

1. Field of the Invention

The present invention relates to a visualizing agent comprising a Janus Green B (JGB), and a visualizing method by using the same. More specifically, the method comprising A method of visualizing the intravascular threadlike structure floating inside a lymphatic vessel comprising injecting JGB into the lymphatic tissue to be stained, and conducting microscopy.

2. Description of the Related Art

In the early 1960's, Bonghan Kim reported finding an intravascular threadlike structure inside lymphatic vessels as part of a large network of a new circulatory system entirely distinct from the vascular or neural systems (9), but kept his method secret, so no one was able to reproduce his results. His work has therefore been neglected, as one might expect, for a long time. Bonghan Kim, a North Korean physiologist, reported his findings on the physical bases of acupuncture meridians, which were named “Bonghan ducts” and supposedly formed a third circulatory system. Through the network of Bonghan ducts flows a liquid that contains special granules that have the power to regenerate cells in damaged tissues. His work was published in five articles from 1962 to 1965 in North Korean medical journals (in Korean). The 9th reference (9) is the only English monograph that has summarized the earlier part of his work.

In an exception to this neglect, the Japanese anatomist Fujiwara was in fact able to partially confirm Kim's results (4) but his work has not attracted much attention either. Only very recently have certain investigators rediscovered the intravascular threadlike structure using a slow perfusion method in rats and rabbits (6) as well as threadlike structures on the internal organ surfaces of rabbits and rats (14, 15, 20). Even though Kim claimed the existence of the threadlike structure inside lymphatic vessels, he did not present any photographic evidence. And as far as we know no one has been able to confirm his claim, including Fujiwara, mainly because Kim's method had not been disclosed.

Since quite early after the discovery of the lymphatic vessel, its anatomical structure and physiological function have been thought to be mostly, if not completely, understood (5, 16). It is firmly established that there is present a flow of liquid, which includes lymphocytes in the lumen of the lymphatic vessel, and no other structure inside the lymphatic vessel is known.

The existence of this novel structure was not noticed previously because it is extremely difficult to detect it by microscopic inspection of lymphatic vessels. In Western studies of anatomy, there is not even the slightest mention of intravascular threadlike structures afloat inside lymphatic vessels (5, 16). The fact that such structures exist on a meso-scale (with thicknesses on the order of 20 μm) and have not been noticed at all in numerous surgical operations is quite a surprise. Undoubtedly, their optical transparency makes them extremely difficult to detect.

### SUMMARY OF THE INVENTION

To resolve the problem that the intravascular threadlike structure floating inside a lymphatic vessel has not been visualized, the inventors of the present invention have found a new method which utilizes JGB, which stained heavily the novel structure so that direct manifestation was achieved. The tissue was studied by confocal laser scanning microscopy (CLSM) obtained by acridine orange staining, light microscopy obtained Hematoxylin, and cryo scanning electron microscopy (Cryo-SEM). Thus it is surprising to discover a threadlike structure afloat inside lymphatic vessels that does not adhere to the vessel wall. It rightly sounds extremely unlikely that threadlike structures of a diameter of about 100 μm could have gone unnoticed despite the many surgeries and studies on the lymphatic vessels throughout the world.

Thus, the object of the present invention is to provide a visualizing agent comprising JGB to vividly exhibit the passage of these structures through lymphatic valves inside lymphatic vessels under stereomicroscope. The discoveries of the dye that strongly stains the novel threadlike tissue and reveals its histological structure are critical contributions toward an important extension of the current understanding of cellular anatomy.

Another object of the present invention is to a visualizing method by using the visualizing agent comprising a JGB.

### BRIEF DESCRIPTION OF THE DRAWINGS

FIG. 1 is a stereoscopic image of lymphatic vessel around the inferior vena cava of a rabbit in accordance with Example 1;

FIG. 2 is optically sectioned serial images of threadlike structure after acridine-orange staining was taken by CLSM in accordance with Example 1;

FIG. 3(A) is a cross sectional image of the threadlike structure (arrow) inside a lymphatic vessel stained by hemtoxylin and eosin (×1,000) with a wide view insertion (×200), and FIG. 3(B) is an oblique sectional image (×400) of the threadlike structure (arrows) with a wide view insertion (×200) in accordance with Example 1;

FIG. 4(A) is the freeze-fractured specimen showing that the collapsed lymphatic vessel (outlined with a dashed line, and which could not be seen in the micrograph) contains another substructure which has a distinctive outer membrane, and FIG. 4(B) is a higher magnification of the rectangle in (A) in accordance with Example 1;

FIG. 5 is cross-sectional images of hemtoxylin and eosin stained specimens, showing lymphatic vessels with a threadlike structure (arrows) under a light microscope (Axiophot, Carl Zeiss, Germany) in accordance with Example 2; and

FIG. 6 is cross-sectional images of a novel threadlike structure inside a lymphatic vessel (outlined with a dashed line) under a cryo-scanning electron microscope (JSM-5410LV, JEOL, Japan) in accordance with Example 2.

### DETAILED DESCRIPTION OF EXEMPLARY EMBODIMENTS

The present invention is described in more detailed hereinafter. The accompanying drawing, which is included to provide further understanding of the invention and is incorporated in and constitutes a part of this specification, illustrates an embodiment of the invention and together with the description serves to explain the principles of the invention.

A visualizing agent used for staining the Bonghan duct inside a lymphatic vessel comprising JGB. The agent is used for visualizing the intravascular threadlike structure in situ and in vivo. The agent visualizes the intravascular threadlike structure by heavier staining than other lymphatic tissue.

The visualizing agent contains 0.01 to 10 (w/v) % of JGB solution, and preferably 0.1 to 5 (w/v) % of JGB solution. The solvent for preparing the JGB solution is particularly not limited, but preferably includes ethyl alcohol, and saline. The more preferable solvent is ethyl alcohol.

In addition, the present invention is related to a method of visualizing the intravascular threadlike structure floating inside a lymphatic vessel using the visualizing agent. More specifically, the present invention is related to a method of visualizing the intravascular threadlike structure floating inside a lymphatic vessel comprising injecting JGB into the lymphatic tissue to be stained, and conducting a microscopy observation. The method of visualizing visualizes the intravascular threadlike structure floating inside a lymphatic vessel in situ or in vivo, which is very important to study the intravascular threadlike structure. The dye JGB can strongly stain the novel intravascular threadlike tissues, which makes it possible to observe them in vivo and in situ inside the transparent lymphatic vessels.

JGB has been known to stain mitochondria and nerves (3, 21). In this invention, the JGB staining method reveals an almost transparent threadlike structure inside the lymphatic vessels of rabbits in situ and in vivo. JGB makes the threadlike structures clearly visible, while several other dyes did not exhibit any such visualization capacity.

A staining method has been developed for in situ and in vivo observation of a threadlike tissue afloat inside the lymphatic vessels of rabbits without adherence to the vessel wall, as if found in normal structures. The existence of this novel structure was not noticed previously because it is extremely difficult to detect it by microscopic inspection of lymphatic vessels. We have found a new method which utilizes Janus Green B, which stained heavily the novel structure so that direct manifestation was achieved. The tissue was studied by a confocal laser scanning microscopy (CLSM), a light microscopy and a cryo scanning electron microscopy (Cryo-SEM). The CLSM image obtained by acridine orange staining of the novel tissue revealed its characteristic nuclei distribution: rod-shaped nuclei of 10-20 μm length aligned in a broken-line/striped fashion. Hematoxylin and eosin staining revealed the threadlike structure passing through a lymphatic valve as histologically distinct from lymphatic vessels and valves. The Cryo-SEM image showed the threadlike structure trapped by a collapsed lymphatic vessel. There were spherical globular structures observable inside sinuses in a rapidly frozen sample, which suggests liquid flowing through the longitudinal ductules in the threadlike structure. The specific staining of the JGB suggests that these threadlike structures inside lymphatic vessels have a high density of mitochondria in their cells and/or nerve-like properties, either of both of which may provide important clues to their physiological function. JGB more effectively made the threadlike structure visible than other dyes employed, i.e.: methylen blue, methyl green and toluidine blue. Those dyes stained the lymphatic vessel as heavily as the threadlike structures, reducing or even obliterating the value of the staining procedure.

Hereinafter, the intravascular threadlike structure floating inside a lymphatic vessel is so called as “Bonghan duct” which Bonghan Kim reported as part of a large network of a new circulatory system entirely distinct from the vascular or neural systems. According to Bonghan Kim's description, the threadlike structures were said to have unique patterns of movement, different from gastrointestinal ducts, and to be parasymphatomimetic (9). This suggests that the Bonghan ducts are stained by JGB because they have a large number of mitochondria, allowing for unique peristaltic movements, and/or have the parasympathetic properties of nerves. This characterization would have deep physiological implications, and will need to be elucidated in the future. The revelation of a high density of mitochondria in the threadlike structure was suggested for the first time in this study. This is an advantage of the current method to the visualizing method using the nanoparticle technique.

The stained the intravascular threadlike structure floating inside a lymphatic vessel is observed with a microscopy. The microscopy is not particularly limited, and microscope being capable of imaging the JGB-stained structure used in conventional art may be used in the present invention. For examples, the microscopy observation can be performed with preferably a stereomicroscope, a light microscopy, CLSM, and a Cryo-SEM. The stereomicroscopy can be performed after injecting a JGB to the lymphatic tissue.

CLSM is performed by observing with a CLSM after acridine orange staining after the injection of a JGB to the lymphatic tissue. In order to prove that the JGB-stained threadlike structure inside lymphatic vessels was not an optical illusion or in any way a visual artifact but rather, real tissue, a piece of the threadlike sample was taken and observed using CLSM after staining by 0.1% (w/v) acridine orange, an agent that characterizes nuclei and their distribution.

For histological study, the light microscopy can be preformed by observing with a light microscope after hematoxyline and eosin staining following the injection of a JGB to the lymphatic tissue. Furthermore, in order to directly demonstrate that there is a novel structure inside the lymphatic vessel, the lymphatic vessel with the threadlike structure was studied using a histological method and cryo-SEM. Cross and oblique sectional images of the tissue show its peculiar structure, and a cryo-SEM image shows a detailed view with liquid inside which was maintained by a rapid freezing of the samples.

The present invention convincingly demonstrates the evident existence of the novel structures. In the earlier work on the intravascular structure of blood vessels, it had been forced to use the complicated processes such as a special perfusion method, an acridine orange fluorescence technique, and hematoxylin and eosin staining to prove the existence and novelty of these newly observed structures (13). Hence, the discovery of the JGB staining technique, and subsequently the in situ and in vivo demonstration method of the novel structure inside lymphatic vessels, was crucial to establish its existence.

The hematoxylin and eosin stained sectional images, and the freeze-fractured sample images obtained by cryo-SEM, show convincingly that certain threadlike structures exist floating inside the lymphatic vessel. The thickness of the threadlike structures varies widely from one subject to another. The thickness may depend upon the functional state of the threadlike structures and/or the physiological state of the subject animal. The diameter and bundle structure, and the size and distribution of endothelial nuclei of the intravascular threadlike structures in various lymphatic vessels, were similar to those found in arteries and veins of rabbits and rats (13). The bundle structure of the threadlike structure composed of ductulus, the size of which is approximately 5 μm, is inferred from the CLSM and histological images. Furthermore, the noticeably spherical globular structures and segregation zones observable in the cryo-SEM image imply that there was a viscous liquid inside the sinuses of the ductulus. These novel threadlike structures were examined in rats with similar results. The results in rats are provided in the example.

With these results, it is reasonable to seriously take into consideration Kim's statements about the physiological function of this structure. His work was intended to establish the physical bases for acupuncture meridians and acupoints. Recently ultrasonic imaging of acupoints has revealed structures in agreement with his claims (7). However, both his theory and its relation to the currently widely used neurophysiological models (2) need to be further investigated. According to Kim, some sort of liquid with granules containing DNA (10) flows through the ducts, which are similar to microcells and might be deeply related to cell-therapy (17). In addition, the threadlike ducts were considered to be deeply related to developmental processes and, in particular, the lymphatic intravascular threadlike structure was related to hematopoietic function (17). Whatever the eventual outcome of deeper investigation of these claims, the finding of the novel structure inside lymphatic vessels is not a mere curiosity but rather a herald of a major breakthrough in cellular anatomy and physiology. Further studies of its histological aspects and physiological functions suggest the possibility of critical new insights in both biology and medicine.

The present invention will now will be described more fully hereinafter with reference to the accompanying examples, in which exemplary embodiments of the invention are shown.

### Example 1

### Threadlike Structures Inside the Lymphatic Vessels of Rabbit

New Zealand White rabbits (females, 12 weeks old) were obtained from the Korean Jung-Ang Lab. Animal Inc. The procedures and the care of the animals conformed to current international laws and policies (Guide for the Care and Use of Laboratory Animals, National Academy Press, 1996). The rabbits were anesthetized with urethane (1.5 g/kg), administered intraperitoneally, and all surgical procedures were performed under general anesthesia.

The abdominal sides of the rabbits were incised, and the large lymphatic vessels beside the inferior vena cava were exposed. In order to stain a threadlike structure afloat inside a lymphatic vessel, approximately 0.3 ml of JGB (dye content 65% Janus Green B (Aldrich, USA) 1 g in 99.9% ethyl Alcohol 100 ml) pre-heated at 40° C. was injected as slowly as possible into a lymph node or lymphatic vessel close to the inferior vena cava using a fine needle of gauge 30½. After the injection needle was carefully withdrawn, the blue color of JGB flowing inside the lymphatic vessel was observed. Then the pre-heated PBS at 40° C. was injected into the lymphatic vessel and lymph node in order to wash out the backward-flowing JGB and to keep them warm and maintain their vital condition. Careful observation was carried out under a stereomicroscope. The position of well-stained threadlike structure in the lymphatic vessel varied according to the staining and lymphatic vessel conditions of the subject rabbits. When the well-stained part was found, the careful removing of connective tissues around the lymphatic vessel was carried out to allow clear observation and sample-taking to produce specimens.

For CLSM observation, the specimens were fixed in 10% neutral buffered formalin as quickly as possible, and then part of them were stained by 0.1% (w/v) acridine orange (Sigma-Aldrich, USA) in PBS for CLSM (LSM510, Carl Zeiss, Germany).

For histological study, the lymphatic vessel with threadlike structures was fixed in 10% neutral buffered formalin for paraffin embedding. Sections of 5 μm thickness were cut with a microtome and stained with hematoxylin and eosin. The sections were observed and photographed under a light microscope (Axiophot, Carl Zeiss, Germany).

For cryo-SEM observation, the sample fixed by the above method was packed into a rivet that was mounted onto a slotted metal stub, and plunged into liquid nitrogen for cryo-fixation for 20 seconds. Frozen specimens were placed in a cryo-chamber of a cryo-transfer system (CT1500, Oxford Instruments, Oxon, UK) maintained at −170° C., and transferred to the cold specimen stage of a scanning electron microscope (JSM-5410LV, JEOL, Tokyo, Japan). The specimen was then freeze-fractured to expose the internal structure of frozen lymphatic vessel containing JGB stained threadlike structure that protruded above the rivet surface in the cryo-chamber using a cold knife. Sublimation of superficial frozen water was performed by heating and maintaining the cold specimen stage at −60° C. for 10 minutes and the process was monitored with the electron microscope at an accelerating voltage of 5 kV. The specimens were sputter-coated with gold (approximately 30 nm in thickness) in the cryo-chamber, and observed with an electron microscope at 20 kV.

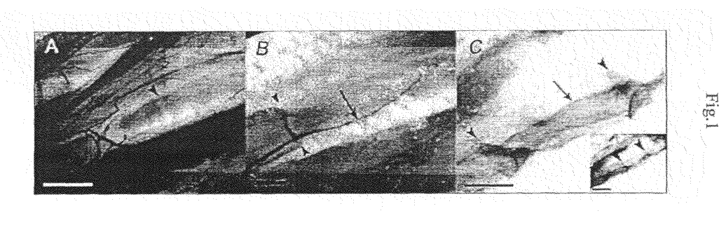

FIG. 1 is a stereoscopic image of lymphatic vessel around the inferior vena cava of a rabbit. (A) typical view without staining and (B, C) after injection of JGB into the lymphatic vessel. In (A), lymphatic valves (arrowhead) inside the lymphatic vessel and blood capillaries around the lymphatic vessel wall are observable. After JGB injection, the threadlike structure (arrow) in (B) is clearly exhibited with a stained lymphatic valve (arrowhead) inside the weakly stained lymphatic vessel wall. The threadlike structure (arrow) in (C) is the biggest one (,) among those observed, whose size is approximately 600 μm in diameter. In the inserted figure, three stained lymphatic valves (arrowheads) indicate the size of the lymphatic vessel and the well-stained novel structure may be seen lying in and through those valves. Scale bars are 1 mm.

A stereomicroscopic view of the lymphatic vessel around the inferior vena cava is shown in FIG. 1A, where the diameter is approximately 600 μm. Notice that blood capillaries on the wall of the lymphatic vessel are clearly visible and even those below the bottom wall of the lymphatic vessel can be seen. One can also notice the lymphatic valve, although not so clearly. However, there was not even a slight hint of any other structure inside the lymphatic vessel. This is a typical situation in that the novel tissue had eluded observation with an ordinary microscope. However, an unexpected structure inside the lymphatic vessel emerged after the staining dye JGB was injected into either the lymphatic vessel or lymph node. FIGS. 1B and 1C are stereoscopic images after the injection of JGB into lymphatic vessel or lymphatic node. The JGB-stained threadlike structure was clearly observable, with a stained lymphatic valve and weakly-stained lymphatic vessel wall, as seen in FIG. 1B. Another very “thick” threadlike structure was observed in a cylindrical form between two lymphatic valves, as in FIG. 1C. The thickness of the threadlike structures also varied widely from one subject to another, as illustrated in FIGS. 1B and 1C. The insert in FIG. 1C shows that a JGB-stained threadlike structure is floating through three stained lymphatic valves inside the weakly-stained lymphatic vessel wall.

FIG. 1B and FIG. 1C are typical of the images obtained during the fourteen experiments presented in Tables 1 and 2. We examined the abdominal lymphatic vessels connected to a lumbar node beside the inferior vena cava and iliac veins, and the lymphatic vessels in the peritoneum around the intestines. During the fourteen experiments, we observed the intravascular structure of the lymphatic vessels from every rabbit except for two failures. The JGB-stained threadlike structure was, therefore, not an abnormal state of one particular subject. The diameters of the lymphatic vessels we examined range from 130 μm to 2000 μm and the diameters of the threadlike tissues range from 26 μm to 500 μm, with an average 129 μm. As one can see in the FIGS. 1B and 1C, the thickness of the threadlike structures varies widely from one subject to another.

Table 1 shows size data (of) on the nineteen threadlike structures from fourteen New Zealand White rabbits. The data format is average±standard deviation. More detailed information is presented in Table 2.

TABLE 1

| Lymphatic vessel position | The number of threadlike | The diameter of the | The diameter of the |
|---|---|---|---|
| where sample is taken | structures | lymphatic vessel (μm) | threadlike structure (μm) |
| around the inferior vena cava | 15 | 785.6 ± 537.9 | 153.5 ± 142.6 |
| On the large intestine | 1 | 180.0 | 13.0 |
| around the iliac vein | 3 | 510.0 ± 340.7 | 43.3 ± 7.0 |
| Total | 19 | 719.7 ± 506.0 | 129.4 ± 134.6 |

TABLE 2

| Subject | | | | Lymph vessel | The diameter of | The diameter of | The length of |
|---|---|---|---|---|---|---|---|
| Date | | Weight | Injection | position where | the lymph | the threadlike | the threadlike |
| (2004) | Sex | (kg) | Site | sample is taken | vessel (μm) | structure (μm) | structure (μm) |
| Oct. 18 | Female | 2.2 | Lymph vessel | the environs of | M: 1500 | 100 | 4000 |
| | | | along caudal | the caudal vena | m: 600 | | |
| | | | vena cava | cava | | | |
| Oct. 25 | Female | 2.2 | Lumbar | the environs of | 2000 | 50 | 3000 |
| | | | node | the caudal vena | | | |
| | | | | cava | | | |
| Nov. 01 | Female | 2.2 | Lumbar | the environs of | 1060 | 80 | 12000 |
| | | | node | the caudal vena | | | |
| | | | | cava | | | |
| Nov. 10 | Female | 2.2 | Lumbar | the environs of | 100 | 16 | 220 |
| | | | node | the caudal vena | 880 | DM: 510 | 4000 |
| | | | | cava | | Dm: 190 | |
| Nov. 11 | Female | 2.2 | Lymph vessel | the environs of | M: 400 | 30 | 5000 |
| | | | along caudal | the caudal vena | m: 180 | | |
| | | | vena cava | cava | M: 880 | 60 | 3200 |
| | | | | | m: 320 | | |
| Nov. 15 | Female | 2.2 | Lumbar node | the environs of | 540 | 80 | 940 |
| | | | | the caudal vena | | | |
| | | | | cava | | | |
| Nov. 16 | Female | 2.2 | Lumbar node | the environs of | X | X | X |
| | | | | the caudal vena | | | |
| | | | | cava | | | |
| Nov. 18 | Female | 2.2 | Lumbar node | the environs of | X | X | X |
| | | | | the caudal vena | | | |
| | | | | cava | | | |
| Nov. 24 | Female | 2.2 | Lumbar node | the environs of | 180 | 50 | 1600 |
| | | | | the caudal vena | 600 | 230 | 2700 |
| | | | | cava | | | |
| Nov. 25 | Female | 2.2 | Lumbar node | the environs of | 1500 | 500 | 5000 |
| | | | | the caudal vena | 1300 | DM: 400 | 1700 |
| | | | | cava | | Dm: 170 | |
| | | | | | 960 | 170 | 6600 |
| Nov. 29 | Female | 2.2 | Lymph vessel | The area above | 360 | 26 | 1180 |
| | | | on the large | (of) the large | | | |
| | | | intestine | intestine | | | |
| Dec. 01 | Female | 2.2 | Lumbar node | the environs of | 130 | 40 | 2700 |
| | | | | the caudal vena | 540 | 260 | 4540 |
| | | | | cava | | | |
| Dec. 06 | Female | 2.2 | Caudal lymph | the environs of | 270 | 44 | 2700 |
| | | | node | the ilic vein | 360 | 50 | 2180 |
| | | | | | | | B: 1800 |
| Dec. 07 | Female | 2.2 | Caudal lymph | the environs of | 900 | 36 | 1640 |
| | | | node | the iliac vein | | | |

Table 2 contains detailed information about the fourteen rabbits of table 1 in the main text. The abbreviations are as follows: M=maximum, m=minimum, B=branch, X=failure to obtain the sample, DM=duct maximum, Dm=duct minimum, JGB=Janus green B

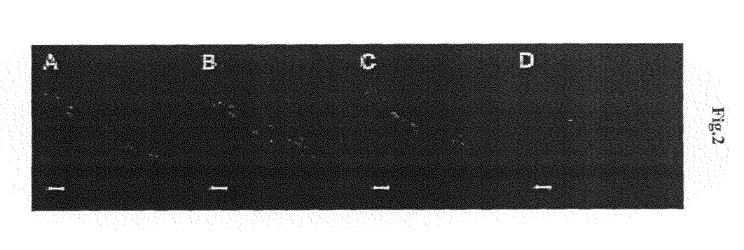

FIG. 2 is optically sectioned serial images of threadlike structure after acridine-orange staining was taken by CLSM. The nuclei are rod-shaped and 10˜20 μm long. They form a broken-line array, and the number of lines varied from A to D, in which the focal depth is increased by 1 μm from the bottom of the threadlike structure. The scale bars are 10 μm.

CLSM observation elucidated the distribution of the nuclei of the threadlike structures. One part of the structure of the threadlike structure was magnified 400 times and optically sectioned by 1 μm. This sectioned image clearly revealed that the threadlike structure contained characteristic rod-shaped nuclei whose lengths are approximately 10˜20 μm, and that these nuclei are arranged in a broken-line striped fashion. The numbers of broken-line stripes vary in FIGS. 2A to D, in which the focal points have been moved by 1 μm one by one from the bottom. These features imply that the threadlike structure could consist of a “bundle” structure consisting of small ductulus the diameter of which is approximately 5 μm. This structure is also consistent with previous reports on similar structures inside blood vessels (1, 13) and on the internal organ-surface of rabbits and rats (14, 15, 20).

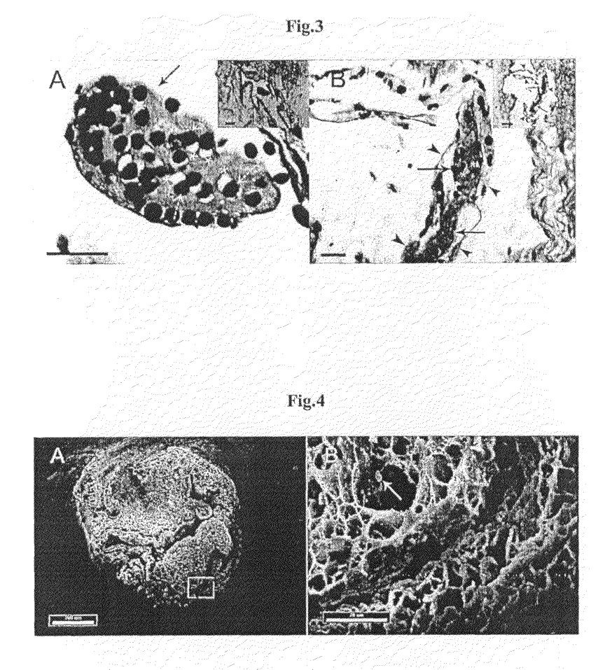

FIG. 3. (A) Cross sectional image of the threadlike structure (arrow) inside a lymphatic vessel stained by hematoxylin and eosin (×1,000) with a wide view insertion (×200). (B) Oblique sectional image (×400) of the threadlike structure (arrows) with a wide view insertion (×200). As seen in the inserted figures, the novel tissue did not adhere to the lymphatic vessel wall and lymphatic valve (arrowheads). There are several big and small sinuses (asterisks) partially surrounded by heavily hematoxylin-stained nuclei (dashed arrow) and these consistently appear at the same site along the threadlike structure. Scale bars are 20 μm, and the scale bars of the inserted figures are 50 μm.

A sectional image was taken, after staining hematoxylin and eosin, to get information on the location of the novel structure inside the lymphatic vessel and on its cells and sinuses. A cross-sectional image shows clearly that there is a distinctive tissue element not attached to the lymphatic vessel wall (see insert of FIG. 3A). FIG. 3A shows that the novel tissue has an outer membrane and contains heavily stained nuclei distinctively different from the endothelial cells in the lymphatic vessel. There were several big and small sinuses in the extra-cellular matrix inside this novel tissue. These sinuses appeared continuously at the same site in several separate slides. The continual appearances of sinuses slide by slide suggest that they are ductulus that require further study along with efforts to elucidate the physiological function of this new tissue. The oblique section in FIG. 3B shows a remarkable image of the threadlike structure mostly surrounded by a lymphatic valve. This image and the inset depict the passage of the threadlike structure through the valve. This striking picture is an indisputable demonstration of the real existence of the threadlike structure.

This novel tissue has characteristic features present in the nuclei as well as the many sinus-like structures in extra-cellular matrix. Beyond the reach of a morphological method like CLSM or the histological work with hematoxylin and eosin staining, cryo-SEM enables a grasp of the liquid intrinsically present in samples as well as cellular structures (18). Soluble materials, organelles, and macromolecules are retained in segregation zones during freezing. This method also revealed the detailed morphology of the threadlike structures inside the lymphatic vessel.

FIG. 4 (A) is the freeze-fractured specimen shows that the collapsed lymphatic vessel (outlined with a dashed line, and which could not be seen in the micrograph) contains another substructure which has a distinctive outer membrane. Fig. (B) Higher magnification of the rectangle in (A). Note that the novel structure ha sinuses with segregation zones and ball-like globular structures. In the sinus fluid material flows with the globular structures (arrow). Scale bar is 200 μm in Fig. (A) and 20 μm in Fig. (B).

In the freeze-fractured specimen, the lymphatic vessel surrounded by connective tissue was found to possess an undulated outer membrane system after freezing followed by sublimation, but the novel tissue was considered to be inside the collapsed lumen of the lymphatic vessel (see FIG. 4A). This novel tissue appears to have a distinct outer membrane that does not collapse, and the dimension of this particular specimen was similar to that found in the stereoscopic image FIG. 1C. As shown in the rectangle of FIG. 4A and its magnified view in FIG. 4B, the sinus in this novel tissue has a wider spacing of segregation zones than the surrounding cytoplasm (FIG. 4B). Even the sinus has segregation zone and spherical globular structures, which implies (that there was) a liquid containing spherical bodies and other components inside the sinus.

### Example 2

### Threadlike Structures Inside the Lymphatic Vessels of Rats

To investigate the morphological and histological studies on those of rats, this example were performed with substantially the same method as Example 1 of the rabbits.

Six Wistar rats (3 males and 3 females) of about 170 grams were obtained from Jung Ang Laboratory Animal Company for use in this study. The animals were housed in a constant temperature-controlled environment (23° C.) with 60% relative humidity, under a 12-h light/dark cycle. All of the animals had ad libitum access to food and water. The procedures involving the animals and their care were in full compliance with current international laws and policies (Guide for the Care and Use of Laboratory Animals, National Academy Press, 1996).

The rats were anesthetized with urethane (1.5 g/kg) administered intraperitoneally, and all surgical procedures were performed under general anesthesia. Under deep anesthesia the abdominal sides of the rats were incised, the node near the inferior vena cava was located (lumbar node), and exposed by removing the surrounding peritonea and fat.

1% Janus green B (JGB) in ethyl alcohol solution (0.05 ml) was injected into the nodes slowly with a needle (30½ gauge). In the rabbit experiments described in the manuscript text, JGB was injected into not only the lymph nodes but also the lymphatic vessels; however, in the rat experiments, JGB was administered only into three kinds of lymph node, (a) an inferior lymph node, an intestinal lymph node and a lumbar lymph node. In comparison with the rabbit's lymphatic vessels, those of rats were too small to allow injection into the lymphatic vessel.

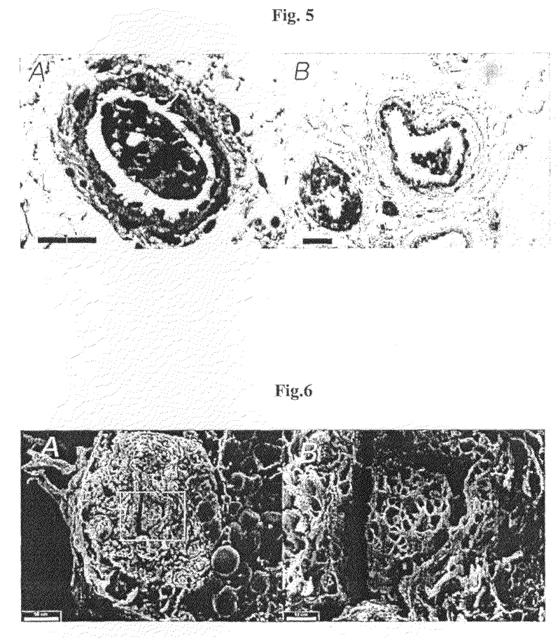

After taking the sample, hematoxylin and eosin staining (FIG. 5), and cryo-scanning electron microscopic observation (FIG. 6) were performed. The results were shown in FIG. 5, FIG. 6 and Table 3.

The results for rats were very similar to those for the rabbits. The average diameter of the threadlike structures in rats is 70±57 μm, which is smaller than those of the rabbits. They are surrounded by a thin outer membrane and contain heavily stained nuclei and extra cellular matrix (FIG. 5). We expect that other mammals also will be found to have the novel threadlike structures inside the lymphatic vessels in a manner similar to these rats and rabbits. Further investigation will be required to test this hypothesis.

FIG. 5 is cross-sectional images of hematoxylin and eosin stained specimens, showing lymphatic vessels with a threadlike structure (arrows) under a light microscope (Axiophot, Carl Zeiss, Germany). The specimens were prepared in a paraffin-section with a thickness of about 5 μm. The novel structure has a thin outer membrane (double line arrows) and contains heavily stained nuclei (dashed arrows) and extra cellular matrix. Also there are several sinuses (asterisks) partially surrounded by nuclei. The ratio of the diameter between the threadlike structure and the lymphatic vessel varies widely from one sample to another. (A) and (B) show the relatively thick and thin threadlike structures, respectively. They are from the same rat but different lymphatic vessels. The different colors of the two images are due to an optical filter. We used a blue filter in (A) to obtain a clear image of the sinuses. The scale bars are 20 μm.

FIG. 6 is Cross-sectional images of a novel threadlike structure inside a lymphatic vessel (outlined with a dashed line) under a cryo-scanning electron microscope (JSM-5410LV, JEOL, Japan). FIG. 6 (A) The freeze-fractured specimens showed that the collapsed lymphatic vessel contains another substructure which had a distinctive outer membrane. Similarly to the case of rabbits the lymphatic vessel collapsed, but the novel structure did not. The scale bar is 50 μm. FIG. 6 (B) A more magnified view of the rectangle in the FIG. 6 (A). We can see the threadlike structure is distinctively separated from the lymphatic vessel wall. This implies that the novel threadlike structure floated in the lymph of the lymphatic vessel. The scale bar is 10 μm. Size data of six threadlike structures from six rats were indicated in the following Table 3.

TABLE 3

| Subject | | | | Lymph vessel | The diameter | The diameter of | The length of |
|---|---|---|---|---|---|---|---|
| | | Weight | Lymph node | position where | of the lymph | the threadlike | the threadlike |
| Date | Sex | (g) | injection | sample is taken | vessel (μm) | structure (μm) | structure (μm) |
| Nov. 18, 2004 | Male | 180 | Caudal lymph | the environs | 340 | 60 | 920 |
| | | | node | of the bladder | B: 90 | B: 20 | B: 680 |
| Nov. 18, 2004 | Female | 160 | Caudal lymph | the lower part | M: 530 | 70 | 5100 |
| | | | node | of lumbar node | m: 210 | | |
| Nov. 20, 2004 | Male | 150 | Intestinal | the environs | 550 | DM: 280 | 3000 |
| | | | node | of the liver | | Dm: 80 | |
| Nov. 22, 2004 | Female | 180 | Caudal lymph | the environs | M: 260 | 50 | 2500 |
| | | | node | of the bladder | m: 100 | | |
| Jan. 10, 2005 | Male | 170 | Lumbar node | the upper part | M: 670 | 50 | 4000 |
| | | | | of lumbar node | m: 290 | | |
| Jan. 19, 2005 | Female | 170 | Lumbar node | the lower part | 130 | 10 | 645 |
| | | | | of lumbar node | | | |

The abbreviations are as follows: M = maximum, m = minimum, B = branch, X = failure to obtain the sample, DM = duct maximum, Dm = duct minimum, JGB = Janus Green B.

### REFERENCES

1. Cho, S. J., Kim, B. S., Park, Y S. 2004. Thread-like Structures in the Aorta and Coronary Artery of Swine. J Int Soc Life Info Sci 22: 609-611.
2. Cho, Z. H., Chung, S. C., Jones, J. P., Park, J. P., Park, H. J., Lee, H. J., Wong, E. K., Min, B. I. 1998. New findings of the correlation between acupoints and corresponding brain cortices using functional MRI. Proc Nat Acad Sci 95: 2670-2673.
3. Cooperstein S. J., Dixit P. K., Lazarow A. 1960. Studies on the mechanism of Janus green B staining of mitochondria. IV. Reduction of Janus green B by isolated cell fractions. Anat Rec. 138: 49-66.
4. Fujiwara, S., Yu S B. 1967. “Bonghan Theory” morphological studies. Igaku no Ayumi 60: 567-577.
5. Hangai-Hoger, N., Cabrales, P., Briceno, J. C., Tsai, A. G. and Intaglietta M. 2004. Microlymphatic and tissue oxygen tension in the rat mesentery. Am J Physiol Heart Circ Physiol 286: H868-H883.
6. Jiang X, Kim H K, Soh K S, et al. 2002. Method for observing intravascular Bonghan duct. Korean J Orient Prevent Med Soc 6: 162-166.
7. Jones, J. P., Bae, Y. K. 2004. Ultrasonic Visualization And Stimulation Of Classical Oriental Acupuncture Points. Medical Acupuncture 15: 24-26.
8. Kellner G. 1966. Bau and Function der Haut. Deutshe Zeitschrift Akupunktur 15: 1-31.
9. Kim, B. H. 1963. On the Kyungrak System J Acad Med Sci DPR Korea 1-41.
10. Kim, B. H. 1965. Sanal Theory. J Acad Med Sci DPR Korea 168: 39-62.
11. Kroger W S. 1973. Acupuncture analgesia: its application by conditioning theory, autogenic training and hypnosis. Am J Psychiatry 130: 855-860.
12. Langevin H M, Yandow J A. 2002. Relationship of acupuncture points and meridians to connective tissue planes. Anat Rec (New Anat) 269B: 257-265.
13. Lee, B. C., Baik, K. Y, Johng, H. M., Nam, T. J., Lee, J., Sung, B., Choi, C., Park, W. H., Park, E. S., Park, D. H., Yoon, Y S., Soh, K. S. 2004a Acridine orange staining method to reveal the characteristic features of an intravascular threadlike structure. Anat. Record 278B: 27-30.
14. Lee, B. C., Park, E. S., Nam T. J., Johng, H. M., Baik, K. Y., Sung, B., Yoon, Y. S., Soh, K. S. 2004b. Bonghan Ducts on the Surface of RAT Internal Organs. J Int Soc Life Info Sci 22: 455-459.
15. Lee, K. J., Kim S., Jung, T. E., Jin D., Kim, D. H., Kim, H. W. 2004. Unique Duct System and the Corpuscle-Like Structures Found on the Surface of the Liver. J int Soc Life Info Sci 22: 460-462.
16. Podgrabinska, S., Braun, P., Velasco, P., Kloos, B., Pepper, M., Jackson, D. G., Skobe M. 2002. Molecular characterization of lymphatic endothelial cells. Proc Nat Acad Sci: 99(25), 16069-16074.
17. Prescott, D. M., Kirkpatrick, J. B. 1973. Mass enucleation of cultured animal cells. In: Prescott D M, editor. Methods in Cell Biol. New York, Academic Press 7: 189-202.
18. Read, N. D. 1991. Low-Temperature Scanning Electron Microscopy of Fungi and Fungus-Plant Interactions. In: Mendgen, K. & Lesemann editors, D. E. Electron Microscopy of Plant Pathogens, Springer, Heidelberg: 17-29
19. Shin, H. S., Johng, H. M., Lee, B. C., Cho, S., Soh, K. S., Baik, K. Y, Yoo, J. S., Soh, K. S. 2005.
20. Feulgen Reaction Study of Novel Threadlike Structures (Bonghan ducts) on the Surface of Mammalian Organs. Anat. Record 284B. in press.
21. Yack J. E. 1993. Janus Green B as a rapid, vital stain for peripheral nerves and chordotonal organs in insects. J. Neurosci. Methods. 49: 17-22.

What is claimed is:

1. A visualizing agent used for staining a Bonghan duct inside a lymphatic vessel which comprises a Janus Green B (JGB).

2. The visualizing agent according to claim 1, wherein the agent is used for visualizing the Bonghan duct in situ or in vivo.

3. The visualizing agent according to claim 2, wherein the agent visualize the Bonghan duct by heavier staining than other lymphatic tissue.

4. The visualizing agent according to claim 1, wherein the agent is a JGB-containing solution.

5. The visualizing agent according to claim 4, wherein the JGB-containing solution includes 0.01 to 10 (w/v) % of JGB in ethyl alcohol or saline.

6. A method of visualizing a Bonghan duct inside a lymphatic vessel which comprises injecting a Janus Green B (JGB)-containing solution into the lymphatic tissue to be stained, and conducting a microscopy observation.

7. The method according to claim 6, wherein the JGB-containing solution includes 0.01 to 10 (w/v) % of JGB.

8. The method according to claim 6, wherein the JGB stains the Bonghan duct in situ or in vivo.

9. The method according to claim 6, wherein the microscopy observation is performed with a stereomicroscope, a light microscope, a confocal laser scanning microscope (CLSM), or cryo scanning electron microscope (Cryo-SEM).

10. The method according to claim 6, wherein the method further comprises hematoxyline and eosin staining of the Bonghan duct after the injection of a JGB to the lymphatic tissue.

11. The method according to claim 10, wherein the microscopy observation is performed with a light microscope.

12. The method according to claim 6, wherein the method further comprises an acridine orange staining of the Bonghan duct after the injection of a JGB to the lymphatic tissue.

13. The method according to claim 12, wherein the microscopy observation is performed with a confocal laser scanning microscope.
